(19) United States
(12) Patent Application Publication
(10) Pub. No.: US 2020/0229845 A1
(43) Pub. Date: Jul. 23, 2020

(54) SURGICAL ACCESS APPARATUS

(71) Applicant: IHSAN TASCI, ISTANBUL (TR)

(72) Inventor: IHSAN TASCI, ISTANBUL (TR)

(21) Appl. No.: 16/641,240

(22) PCT Filed: Oct. 13, 2017
(86) PCT No.: PCT/TR2017/050496

§ 371 Date: Feb. 23, 2020

(51) Int. Cl. A61B 17/34; A61B 90/57

(52) U.S. Cl. CPC A61B 17/3423; A61B 90/57; A61B 2017/349; A61B 2017/3419; A61B 2017/3447

(57) ABSTRACT

The present invention provides a surgical access apparatus for minimally invasive surgery comprising a surgical access device having a port body with one or more passages there through for passing surgical instruments through the surgical access device; and a carrying structure having a first arm extending from the surgical access device to locate the surgical access device.

### TECHNICAL FIELD OF THE INVENTION

The present invention relates to a surgical access apparatus for use in minimally invasive surgical procedures such as endoscopic or laparoscopic surgical operations.

### BACKGROUND OF THE INVENTION

In order to reduce the trauma to the patient and recovery time, many surgical operations are performed through small incisions in the skin compared to the larger incisions required in traditional procedures. Such procedures are referred to as “endoscopic” or “laparoscopic” when performed on the abdomen. The term “minimally invasive” encompasses all of those procedures.

During a minimally invasive operation, surgical tools and devices including endoscopes are inserted into the patient's body through the incision in the tissue. In order to facilitate the accessibility of a tissue and to maintain a fluid tight seal of the incision to prevent insufflations from the surgical site, surgical access devices are known comprising valves and seals and passages, which are placed into the incision.

Performing a surgical operation utilizing a single incision is advantageous. However, the placement of a plurality of cannulas and laparoscopic instruments next to each other through the same small incision creates interference between the instruments that reduces the surgeon's ability to perform the surgery. There are surgical access ports employing joint like mechanisms in order to control the surgical instruments passed through them more precisely. However, the inventor of the present invention determined that the conditions of the incision region prevent a smooth operation of the joint like mechanisms employed in plastic ports. Especially, contractions of the muscles and the tissue surrounding the incision region obstruct the use of surgical instruments extending through the joint like mechanisms.

Hence, a need exists for surgical access devices that provide better stabilization and high precision maneuverability of the surgical tools during a surgery and that reduce the interference between the surgical tools during a minimally invasive surgery preferably through a single incision.

Accordingly, an object of the present invention is to provide a support structure for a surgical access device enabling high precision control of the surgical instruments passed through the surgical access device during a minimally invasive surgery.

### SUMMARY OF THE INVENTION

In order to overcome the above mentioned drawbacks of known devices, the present invention provides a surgical access apparatus for minimally invasive surgery comprising a surgical access device having a port body with one or more passages there through for passing surgical instruments through the surgical access device; and a carrying structure having a first arm extending from the surgical access device to locate the surgical access device.

One or more passages are arranged for passing surgical instruments through the surgical access device into the body of the patient during the minimally invasive surgery, wherein the carrying structure is arranged to locate the surgical access device with respect to the body. The first arm extends external of the surgical access device and is arranged to support the surgical access device externally. The surgical access device is adapted to be placed in an incision in the body to secure an access opening for the surgical instruments.

The carrying structure may comprise a stationary frame and/or mountable to a stationary structure, especially to a surgery table. Hence, the carrying structure may be arranged to connect the surgical access device to the stationary structure, especially to the surgery table. The stationary frame may be set on or below the body of the patient to fix the carrying structure.

The surgical access apparatus of the present invention enhances the stabilization of the surgical access device and the surgical instruments that are passed through the one or more passages of the surgical access device. Therefore, a first end of the first arm may be, preferably pivotally, connected to a connection element of the carrying structure, which is preferably mountable to a surgery table to fix the surgical access device at the body of the patient.

Hence, according to an aspect of the invention, the port body and the surgical instruments are supported by the first arm at the first end thereof.

Present invention provides a surgical access device for minimally invasive surgery comprising a port body having one or more passages therethrough for passing surgical instruments through the surgical access device into the body of the patient, wherein a joint is arranged in the port body in at least one of the one or more passages for pivotally supporting the surgical instruments within the port body and wherein the surgical access device further comprises a hollow sleeve encasing the port body, wherein the sleeve is made of a material having more rigidity than a material of the port body.

Hence, according to an aspect of the invention, the port body and the instruments therein are carried by a self supporting structure of the sleeve.

According to the invention, the sleeve is made more rigid than the port body in order to be able to withstand pressure due to forces exerted by the muscles of patient. Preferably the sleeve is made of metal, more preferably the port body may be made of plastic. The hollow sleeve made of metal is formed to removably accommodate the port body such that the port body may be replaced after each surgery. Such a configuration provides advantages due to reusability of the metallic sleeve portion.

In order to conform to the incision, the sleeve may comprise a removable seal layer that is deformable and/or inflatable and less rigid than the sleeve and enclosing the sleeve at least partially for sealing between the body of the patient and the surgical access device. The seal layer may be made of plastic. The seal layer may be formed integral with the port body preferably substantially concentrically, wherein the sleeve is arranged between the seal layer and the port body.

The port body comprises a support layer having at least a passage defined by a circumferential passage wall extending between a first circumferential edge of a first opening at a first side of the support layer and a second circumferential edge of a second opening at an opposite second side of the support layer and a tube, preferably in form of a cannula extending through the passage at least from the first side of the support layer for passing a surgical instrument through the surgical access device into the body of the patient, wherein the cannula is arranged spaced apart at least from the first edge such that a spacing is provided between the cannula and the passage wall so that the cannula is rotatable within the passage without compressing the support layer. Preferably, the cannula extends further through the passage from the second side of the support layer, wherein the cannula is arranged spaced apart further from the second edge. Preferably, the joint is provided between the cannula and the passage wall to locate the cannula spaced apart from the passage wall, first edge and/or second edge. Preferably, the joint may be integrally formed with or installed onto the cannula or into the passage wall.

According to the invention, the cannula is located spaced apart from the first and/or second edge of the passage wall until it is fully rotated from an un-rotated state, where it is substantially vertical to the support layer or substantially parallel to the passage, to a fully-rotated state, whereby the rotation of the cannula is blocked by the first and/or second edge. A rotation limitation of the cannula may be adjusted by adapting the size of a minimum spacing distance between the cannula and the first and/or second edge. The cannula may rotate about an axis extending substantially parallel to the support layer.

According to the invention, the joint may comprise a ball joint having a channel there through and rotatably arranged in a corresponding seat formed by the passage wall. The ball joint may be provided on the cannula such that it rotates together with the cannula. Hence, the surgical instrument passes through the joint into the body.

According to the invention, the port body comprises preferably a plurality of cannulae or at least two cannulae.

The tube or cannula may extend from the first side and/or the second side of the support layer, wherein the sleeve may completely accommodate the tube or cannula at least at one of the first side and second side of the support layer to protect the inner parts and organs of the body from a possible damage and to provide a better sight to the surgeon. Hence, the tube or cannula may not extend out of the sleeve in to the body at the first side and/or the second side.

According to the invention, the support layer may be made from a stiff material e.g. polycarbonate material or a material having equivalent or similar toughness.

The port body may further comprise a cover layer covering the support layer at least partially at the first and/or second side and made from a softer material than the support layer or the port body, wherein the cannula extends through the cover layer at the first and/or second side of the support layer. The cover layer may be compressed and deformed by the rotation of the cannula.

The surgical access device may further comprise a circumferential intermediate layer provided between the sleeve and the port body to support the port body within the sleeve. The outer surface of the sleeve may be formed threaded in order to ease the installation of the surgical access device in to the incision.

According to the invention, the sleeve or the port body may comprise a releasable connection means, preferably arranged externally and/or at an end portion or at the rim at the first side of the support layer, preferably circumferentially, to establish a releasable coupling between the carrying structure and the sleeve. Alternatively, the sleeve may be formed integral with the first arm. In this case, the sleeve and the carrying structure first arm may be connected via a first joint.

Preferably, a second end of the first arm may comprise a connection member, clips or a gripper to grip the sleeve at its outer peripheral surface via the releasable connection means. The releasable connection means on the outer peripheral surface of the sleeve may comprise an engaging member, preferably a protrusion, a groove, a rib or a rail to be engaged by the gripper. The gripper may comprise a rib, rail or a groove respectively cooperating with the engaging member.

According to a further aspect of the invention, the carrying structure may comprise a second arm for holding a surgical instrument, preferably an endoscope, movable with respect to the clips or gripper and/or positionable above the clips or gripper such that the surgical instrument supported by the second arm can be passed through the surgical access device into the body of the patient through one of the passages. The carrying structure and the rigid sleeve enhance thereby the stabilization of the endoscope within the body. A first end of the second arm may be articulated on the first arm, preferably between the first and second ends. A second end of the second arm may comprise a, preferably articulated, attachment part or a connection member for supporting the surgical instrument. The first end of the second arm may be connected with the first arm over a joint.

The present invention also provides a carrying structure for a surgical access device with one or more passages having a first arm with a first end and an opposite second end comprising means to support the surgical access device at the second end thereof and a second arm with a first end and an opposite second end articulated on the first arm for holding a surgical instrument at the second end thereof movable above the surgical access device such that the surgical instrument supported by the second arm can be passed through the surgical access device into the body of the patient through one of the passages.

The first arm may comprise a connection member at the second end thereof to connect to the surgical access device. The first arm may comprise means to be mounted to a stationary structure at the first end thereof.

The second arm may be articulated at the first end thereof on the first arm, preferably between the first and second ends of the first arm. The second end of the second arm may comprise a, preferably articulated, attachment part or a connection member for supporting the surgical instrument. The first end of the second arm may be connected with the first arm over a joint.

Means to be mounted and/or articulated parts throughout the description of the invention may comprise joints and/or fixation members to lock the articulations and fix the positions of the connected parts with respect to each other.

These and further advantages of the current invention are disclosed in the appended claims.

### DESCRIPTION OF THE DRAWINGS

The above disclosed and further features of the current invention will be better understood with the following detailed description and drawings of the preferred embodiments of the invention.

FIG. 1 shows a preferred embodiment of a surgical access device according to the present invention;

FIG. 2 shows a cross sectional view of the surgical access device according to the present invention;

FIG. 3 shows a preferred embodiment of the surgical access apparatus according to the present invention;

FIG. 4 shows a general view of the preferred embodiment of the surgical access apparatus according to the present invention;

FIG. 5 shows a further general view of the preferred embodiment of the surgical access apparatus according to the present invention;

FIG. 6 shows a further preferred embodiment of the surgical access apparatus according to the present invention.

### DESCRIPTION OF PREFERRED EMBODIMENTS

The preferred embodiments of the current invention will be described with references to the appended drawings. Equivalent features of the preferred embodiments have been denoted with the same reference signs throughout the description and the definitions of such features are not repeated.

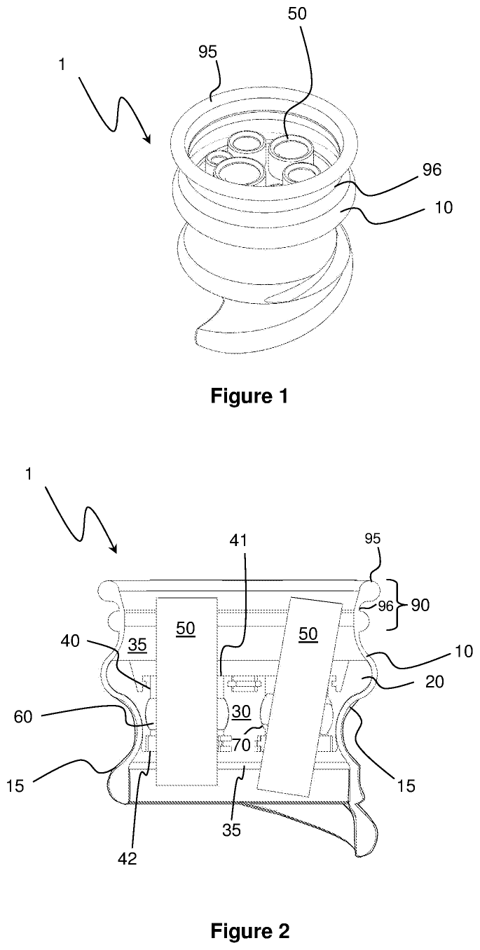

The surgical access device 1 of a preferred embodiment of the invention shown in FIG. 1 and as a cross section in FIG. 2 comprises a hollow sleeve 10 to be placed in an incision on the body of a patient and a port body 20 removably arranged in the hollow sleeve 10, wherein the port body 20 comprises a support layer 30 having passages defined by circumferential passage walls 40 and cannulae 50 extending through the passages respectively for passing surgical instruments into the body of the patient during surgery, wherein the cannulae 50 are arranged spaced apart from the respective passage walls such that the cannulae 50 are pivotally arranged within the respective passages. Alternatively, the port body 20 may comprise a single cannula 50 or at least two cannulae 50.

In order to conform to the incision, the sleeve 10 comprises an optional removable seal layer 15 that is deformable and less rigid than the sleeve 10 and that encloses the sleeve 10 partially for sealing between the body of the patient and the surgical access device 1. The seal layer 15 is made of plastic. The seal layer 15 is formed integral with the port body 20 substantially concentrically, wherein the sleeve 10 is inserted between the seal layer 15 and the port body 20. Alternatively, the seal layer 10 may be installed onto the sleeve 10 as a separate part. The seal layer 15 may also be inflatable.

The port body 20 comprises ball joints 60 arranged on the cannulae 50, between the cannulae 50 and the passage walls 40. Each ball joint 60 is rotatably arranged in a corresponding seat 70 formed in the support layer 30 by the passage wall 40. The ball joints 60 are provided on the cannulae 50 such that they rotate together with the cannulae 50.

The support layer 30 of the preferred embodiment is made from polycarbonate material or a material having equivalent or similar toughness.

The circumferential passage walls 40 extend between a first circumferential edge 41 of a first opening at a first side of the support layer 30 and a second circumferential edge 42 of a second opening at an opposite second side of the support layer 30. The cannulae 50 extend through the passages from the first and the second side of the support layer 30. In this preferred embodiment, the ball joint 60 is installed to the cannula.

The cannula 50 is located spaced apart from the first and/or second edge 41, 42 of the passage wall 40 until it is fully rotated from an un-rotated state, where it is substantially vertical to the support layer 30 or substantially parallel to the passage, to a fully-rotated state, whereby the rotation of the cannula 50 is blocked by the first and/or second edge 41, 42. A rotation limitation of the cannula 50 may be adjusted by adapting the size of a minimum spacing distance between the cannula 50 and the first and/or second edge 41, 42. The cannulae 50 may rotate about an axis extending substantially parallel to the support layer 30. Alternatively, one or more tubes may be used instead of the cannulae 50, or the ball joint 60 may comprise a channel through a surgical instrument may be passed.

In this embodiment, the sleeve 10 completely accommodates the cannulae 50 at the second side of the support layer 30 to protect the inner parts and organs of the body from a possible damage and to provide a better sight to the surgeon.

In this embodiment, the sleeve is made of metal in order to be able to withstand pressure due to forces exerted by the muscles of patient and the port body 20 is made of plastic in order to enable easy mounting into the sleeve. Hence, the hollow sleeve 10 is formed to removably accommodate the port body 20 such that the port body 20 may be replaced after each surgery.

The port body comprises a cover layer 35 covering the support layer 30 at least partially at the first and second side that is made from a softer material than the support layer 30 or the port body 20, wherein the cannula 50 extend through the cover layer 35 at the first and/or second side of the support layer 35. The cover layer 35 may be compressed and deformed by the rotation of the cannulae 50.

The surgical access device 1 further comprises a circumferential intermediate layer (not shown) between the sleeve 10 and the port body 20 to support the port body 20 within the sleeve 10. The outer surface of the sleeve 10 may be formed threaded in order to ease the installation of the surgical access device 1 into the incision.

The sleeve 10 of this preferred embodiment comprises a releasable connection means 90 to establish a releasable coupling between a carrying structure 100 and the sleeve 10. The releasable connection means 90 on the outer peripheral surface of the sleeve 10 comprises an engaging member formed in form of a circumferential protrusion 95 and groove 96.

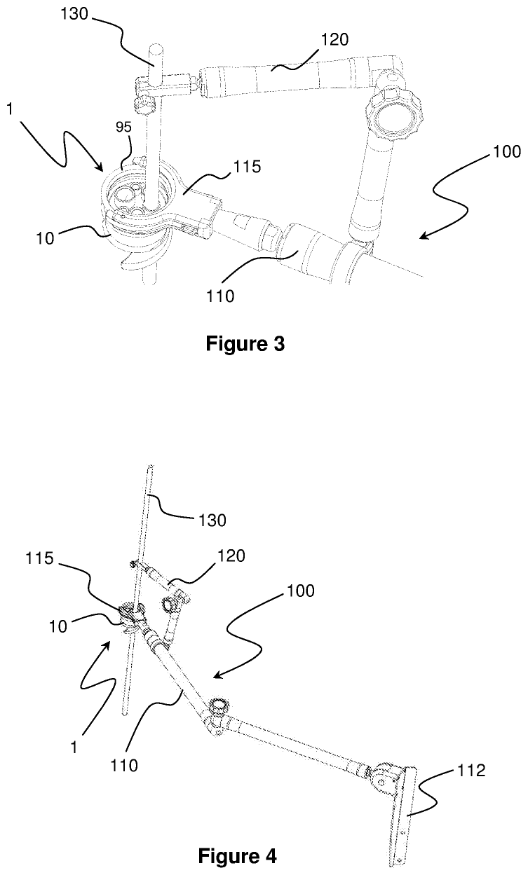

FIGS. 3 and 4 show a preferred embodiment of a surgical access apparatus according to the present invention, according to which a surgical access device 1 to be placed in an incision on the body of a patient comprises passages for passing surgical instruments into the body of the patient during surgery. The surgical access apparatus comprises further a carrying structure 100 having a first arm 110 to fix the surgical access device 1 for enhancing the stabilization of the surgical access device 1 and the surgical instruments that are passed through them.

Therefore, a first end of the first arm 110 pivotally connects to a connection element 112 of the carrying structure 100 attachable to a surgery table to fix the surgical access device 1. The sleeve 10 and the port body 20 of the surgical access device 1 of the second preferred embodiment is formed similar to the first preferred embodiment as described above and therefore they are not described here in detail; however other known surgical access ports may also be used, e.g. a surgical access port having a unitary body i.e. without a sleeve and/or without ball joints.

According to this embodiment, the surgical access device 1 comprises an engaging member in form of a circumferential protrusion 95 and a groove 96 arranged outside of the sleeve 10 at the rim as a releasable connection means 90 to establish a releasable coupling between the carrying structure 100 and the sleeve 10. Alternatively, the sleeve 10 may be formed integral with the first arm 110. A second end of the first arm 110 comprises a gripper 115 to grip the sleeve 10 at its outer peripheral surface via the releasable connection means 90. If a surgical access device without a sleeve should be employed, the engaging member may be formed at the port body or the port body may be formed integrally with the first arm accordingly.

The carrying structure 100 comprises a second arm 120 for holding a surgical instrument, here an endoscope 130. The second arm 120 is movable with respect to the gripper 115 and positionable above the gripper 115 to pass and move the endoscope 130 supported by the second arm 120 through the port body 20 into the body of the patient through one of the passages. The carrying structure 100 enhances thereby the stabilization of the endoscope 130. A first end of the second arm 120 is articulated on the first arm 110 between the first and second ends. The first end of the second arm 120 is coupled to the first arm over a joint mechanism to make the second arm 120 movable with respect to the first arm 110. The second end of the second arm 120 comprises an articulated attachment part for holding the endoscope 130. Alternatively, the second arm 120 may be supported at the connection element 112.

The first arm 110 and the second arm 120 comprise each two arm portions connected via fixable articulation elements to make the endoscope 130 and the surgical access device repositionable with respect to each other and the surgery site on the body of the patient. Other suitable configurations, e.g. bendable first arm and second arm, are also possible according to the specific instrumentation. In use, the carrying structure is installed to a fixed structure such as a surgery table at the first end of the first arm 110.

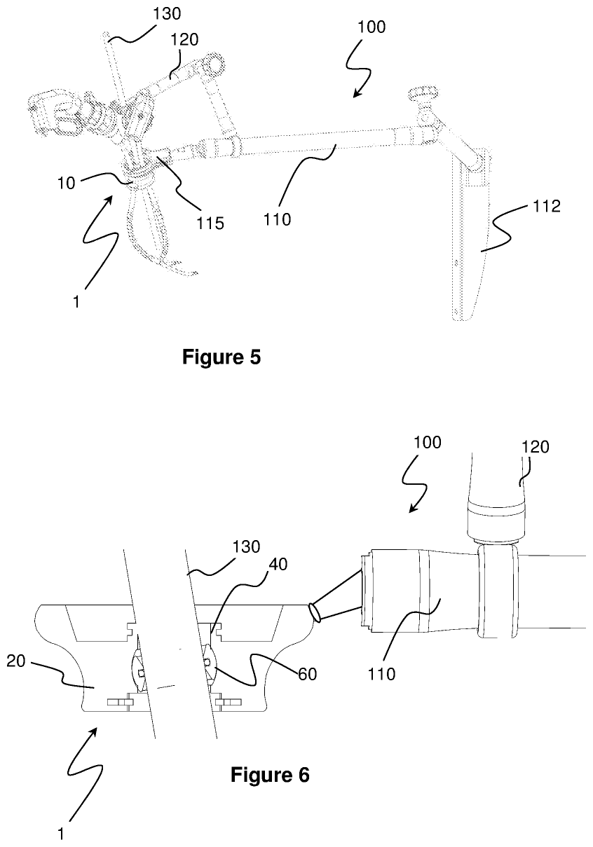

FIG. 5 shows a general view of this preferred embodiment during use with surgical instruments, whereby the gripper 115 has gripped the sleeve 10 at its periphery and the surgical instruments and the endoscope 130 are passed through the passages of the port body 20. The stabilization of the endoscope 130 is achieved by the first arm 110, the second arm 120 and the carrying structure 100 as a whole.

FIG. 6 shows a further preferred embodiment of the present invention, whereby the surgical access device 1 comprises just one passage through which an endoscope 130 passes. The surgical access device has a port body 20 which is directly connected to a first arm 110 of a carrying structure 100 which is attached at a surgery table at a first end (not shown). The carrying structure 100 supports the port body 20 at a second end thereof and a second arm 120 is supported movably on the first arm 110 for pivotally supporting the endoscope 130 above the port body 20. Accordingly, the second arm 120 has joints between its first and second ends and at the connection with the first arm 110. The second arm 120 may also be made bendable using appropriate material and/or mechanisms. The second end of the first arm 110 is articulated on the surgical access device 1.

The design features of the device, such as the number and the location of the cannulae, size and orientation of the parts and joint types and materials, may all be adapted according to different cases appropriately.

The preferred embodiment of the invention is described above in the Drawings and Description of Preferred Embodiments. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s). The foregoing description of a preferred embodiment and best mode of the invention known to the applicant at the time of filing the application has been presented and is intended for the purposes of illustration and description. It is not intended to be exhaustive or to limit the invention to the precise form disclosed, and many modifications and variations are possible in the light of the above teachings. The embodiment was chosen and described in order to best explain the principles of the invention and its practical application and to enable others skilled in the art to best utilize the invention in various embodiments and with various modifications as are suited to the particular use contemplated.

1. A surgical access apparatus for minimally invasive surgery comprising:
a surgical access device comprising a port body with one or more passages therethrough for passing surgical instruments through the surgical access device, characterized by further comprising:
a carrying structure mountable to a stationary structure during the surgery to connect the surgical access device to the stationary structure, wherein the carrying structure comprises a first arm having a first end and a second end opposite to the first end, and wherein the first arm comprises means to be mounted to the stationary structure at the first end thereof and wherein the first arm is attached to the surgical access device at the second end thereof.

2. The surgical access device according to claim 1, wherein a joint is arranged in at least one of the one or more passages for pivotally supporting the surgical instruments, and/or the surgical access device further comprises a hollow sleeve encasing the port body, wherein the sleeve is made of a material having more rigidity than a material of the port body.

3. The surgical access device according one of claim 1 or 2, wherein the sleeve comprises a releasable connection means, preferably arranged externally and/or at an end portion or at the rim of the sleeve.

4. The surgical access device according to claim 1 or 2, wherein the sleeve is formed integral with the first arm at the second end thereof.

5. The surgical access device according one of claims 1 to 3, wherein the second end of the first arm comprises a connection member, preferably a clips or a gripper, to grip the sleeve at its outer peripheral surface via the releasable connection means, which preferably comprises an engaging member, preferably a protrusion, a groove, a rib or a rail to be engaged by the gripper, which preferably comprises a rib, rail or a groove respectively cooperating with the engaging member.

6. The surgical access device according one of claims 1 to 5, wherein the carrying structure comprises a second arm for holding a surgical instrument, preferably an endoscope, movable with respect to the surgical access device and/or positionable above the surgical access device such that the surgical instrument supported by the second arm can be passed through one of the passages.

7. The surgical access device according one of claims 1 to 6, wherein a first end of the second arm is articulated on the first arm and/or the a second end of the second arm comprises means, preferably a connection member, clips or gripper, to hold a surgical instrument.

8. A carrying structure for a surgical access device with one or more passages having a first arm with a first end and an opposite second end comprising means to support the surgical access device at the second end thereof and a second arm with a first end and an opposite second end articulated on the first arm for holding a surgical instrument at the second end thereof movable above the surgical access device such that the surgical instrument supported by the second arm can be passed through the surgical access device through one of the passages.

9. The carrying structure according to claim 8, wherein the second arm is articulated at the first end thereof on the first arm, preferably between the first and second ends of the first arm, preferably via a joint.

10. The carrying structure according to claim 8 or 9, wherein the second end of the second arm comprises a, preferably articulated, attachment part or a connection member for supporting the surgical instrument.

11. The carrying structure according to one of claims 8 to 9, wherein the first arm comprises means to be mounted to a stationary structure at the first end thereof.

12. The carrying structure according to one of claims 8 to 9, wherein the first arm and/or the second arm comprises a plurality of arm portions bendably or pivotably connected to each other.

13. A surgical access device for a surgical access apparatus having a carrying structure according to one of claims 1 to 7, comprising a port body having one or more passages therethrough for passing surgical instruments, characterized in that the surgical access device comprises a releasable connection means arranged externally to attach to the carrying structure.

14. The surgical access device according claim 13, wherein the releasable connection means comprises an engaging member, preferably a protrusion, a groove, a rib or a rail.

15. The surgical access device according claim 13 or 14, wherein a joint is arranged in at least one of the one or more passages for pivotally supporting the surgical instruments, wherein the surgical access device further comprises a hollow sleeve encasing the port body, wherein the sleeve is made of a material having more rigidity than a material of the port body for enhancing the stabilization of the surgical instruments.

16. The surgical access device according to one of claims 13 to 15, wherein the sleeve is made of metal, and preferably the port body is made of plastic.

17. The surgical access device according to one of claim 13 or 16, wherein the sleeve is formed to removably accommodate the port body such that the port body may be replaced.

18. The surgical access device according to one of claims 13 to 17, wherein the sleeve comprises a seal layer enclosing the sleeve, which is removable and/or deformable and/or inflatable and/or which is less rigid than the sleeve.

19. The surgical access device according to one of claims 13 to 18, wherein the port body comprises a support layer having at least a passage and a tube, preferably in form of a cannula extending through the passage for passing a surgical instrument through the surgical access device into the body of the patient.

20. The surgical access device according to one of claims 13 to 19, wherein the joint is provided between the cannula and the passage wall, preferably wherein the joint is integrally formed with or installed onto the cannula or into the passage wall.

21. The surgical access device according to one of claims 13 to 20, wherein the joint comprises a ball joint having a channel there through and rotatably arranged in a corresponding seat formed by the passage wall.

22. The surgical access device according to one of claims 13 to 21, wherein the tube or the cannula extends from the first side and/or the second side of the support layer, wherein the sleeve completely accommodates the tube or cannula at least at one of the first side and second side of the support layer.
